(19) United States
(12) Patent Application Publication
(10) Pub. No.: US 2009/0048561 A1
(43) Pub. Date: Feb. 19, 2009

(54) INJECTION DEVICE WITH AN AXIALLY OVERLAPPING DOSE SETTING OR DISPLAY ELEMENT

(76) Inventors: Stefan Burren, Bremgarten (CH); Philippe Kohlbrenner, Kaltacker (CH); Juergen Wittmann, Burgdorf (CH); Martin Wittwer, Wyssachen (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/142,523

(22) Filed: Jun. 19, 2008

Related U.S. Application Data
(63) Continuation of application No. PCT/CH2006/000673, filed on Nov. 30, 2006.

(30) Foreign Application Priority Data
Dec. 20, 2005 (DE) DE102005060928.7

(51) Int. Cl. A61M 5/315; A61M 5/20

(52) U.S. Cl. 604/135; 604/208

(57) ABSTRACT

An injection device including a housing with a reservoir for an injectable product, a plunger axially movable in a direction of propulsion in the reservoir, a dosing and propulsion mechanism provided for the plunger and arranged in an axial continuation of the reservoir, with a dosing or display member moveable in one axial direction relative to the plunger when setting a product dose and moveable in the opposite direction when the dose is dispensed, and, optionally, a force-imparting member which forces the dosing or display member in the direction of an axial end position, wherein at least one of the dosing or display member and optional force-imparting member axially overlap the reservoir, at least when the dosing or display member is in the end position.

### CROSS-REFERENCE TO RELATED APPLICATIONS

This application is a continuation of International Patent Application No. PCT/CH2006/000673 filed Nov. 30, 2006, which claims priority to German Patent Application No. DE 10 2005 060 928.7 filed Dec. 20, 2005, the contents of both of which are herein incorporated by reference.

### BACKGROUND

The present invention relates to devices for delivering, injecting, administering, infusing or dispensing a substance, and to methods of making and using such devices. In some embodiments, the present invention relates to an injection device for an injectable product, e.g. a liquid medicament such as insulin, a growth hormone, an osteoporosis preparation, etc.

Patent specification EP 1 351 732 B1 discloses an injection device with a housing, in which a reservoir for an injectable product and, accommodated in the reservoir, a dose setting and forward drive unit for a conveyor plunger are disposed. The dose setting and forward drive unit comprises a dose setting element which can be rotated and axially displaced in a thread engagement with the housing to set a product dose. The dose setting element engages a plunger rod in a rotational lock, which permits relative axial movements between the plunger rod and the dose setting element. The dose setting and forward drive unit also has a force transmitting element in the form of a compression spring, which biases the dose setting element axially by an elastic force. The force transmitting element is tensed when the product dose is increased during the setting operation. The plunger rod sits in a thread engagement with the housing and is retained so that it can not rotate relative to the housing when the product dose is being set. The dose setting element simultaneously also serves as a display element for displaying the set dose. The threads of the dose setting element and housing forming part of the thread engagement have a thread pitch which is large enough so that when axial pressure is applied to the dose setting element, it rotates in said thread engagement and is not retained by self-locking. Due to the correspondingly long axial setting path, the markings of the dose scale can likewise be distributed across a correspondingly long length, which is beneficial to enabling a reading to be taken. The disadvantage, however, is that the known injection device is of a correspondingly long length. A trigger for activating the dispensing of the set dose is disposed in an axially central region of the housing. This makes it more difficult to manipulate the injection device during the injection, namely to trigger the dispensing operation.

In another injection device known from patent specification DE 198 21 934 C1, a dose setting element is disposed in the reservoir so that it is not able to move, thereby enabling the installation-length to be reduced. The dose setting element is provided with several stops, each one of which restricts the stroke of a plunger rod during dispensing operations which can be effected one after the other and thus defines the dose which can be administered with each dispensing operation. However, the individual doses can not be freely selected as is the case with the dose setting and display element disclosed in EP 1 351 732.

### SUMMARY

One object of the present invention is to provide an injection device with an axially displaceable dose setting and/or display element of an ergonomically conducive design with as long as possible an axial displacement path.

In one embodiment, the present invention comprises an injection device comprising a housing with a reservoir for an injectable product, a plunger axially movable in a direction of propulsion in the reservoir, a dosing and propulsion mechanism for the plunger and arranged in an axial continuation of the reservoir, a dosing and/or display member moveable in one axial direction relative to the plunger when setting a product dose and moveable in the opposite direction when the dose is dispensed, and, optionally, a force-imparting member which forces the dosing and/or display member in the direction of an axial end position, wherein at least one of the dosing and/or display member and optional force-imparting member axially overlap the reservoir at least when the dosing or display member is in the end position.

In one embodiment, the present invention comprises an injection device comprising a housing with a reservoir for an injectable product, a plunger axially movable in the reservoir in a direction of propulsion, a dosing and propulsion driver provided for the plunger and arranged in an axial continuation of the reservoir, an element, comprising at least one of a dosing member and display member, said element moveable in one axial direction relative to the plunger when setting a product dose and in the opposite direction when the dose is dispensed, and a force-imparting member which forces the element in the direction of an axial end position, wherein at least one of the element and force-imparting member axially overlaps the reservoir, at least when the element is in the end position. In one embodiment, the injection device further comprises a manually operable operating knob which can be moved, by a rotating movement, backward and forward relative to the housing for setting the dose, the knob being kinematically coupled to the element to be prevented from rotating.

In one embodiment, the present invention comprises an injection device comprising a housing with a reservoir for an injectable product, a plunger which is axially movable in a direction of propulsion in the reservoir, a dosing and propulsion means provided for the plunger and arranged in an axial continuation of the reservoir, with a dosing or display member which is moved in one axial direction relative to the plunger when setting a product dose and is moved in the opposite direction when the dose is dispensed, and, optionally, a force-imparting member which forces the dosing or display member in the direction of an axial end position, wherein at least one out of the dosing or display member and optional force-imparting member axially overlaps the reservoir, at least with the dosing or display member in the end position.

In one embodiment, the present invention comprises an injection device comprising a housing with a reservoir for an injectable product, a plunger axially displaceable in the reservoir in a forward drive direction and a dose setting and forward drive unit, which is disposed in an axial extension of the reservoir, i.e. along a displacement axis of the plunger adjacent to the reservoir. The housing may serve directly as the reservoir but, in some embodiments, the housing merely constitutes a holder for a product container, which may be an ampoule with the plunger accommodated in it. The dose setting and forward drive unit has a dose setting or display element, which may assume the function of only dose setting element, only display element, or a dose setting and display element in combination. (The word “or” is used in the sense of “and/or,” i.e. in the logical sense with reference to the dose setting or display element and otherwise, unless otherwise stated in the respective context.) The dose setting or display element can be moved backward and forward axially relative to the plunger and reservoir, and moves in an axial direction relative to the plunger when a product dose to be administered is being set, and moves back in the opposite direction as the dose is being dispensed. In some preferred embodiments, the ability to move axially is restricted by a stop predefining an axial end position as far as which the display element can be moved for at least one of the two axial directions. In some preferred embodiments, an axial end position is predefined by a contact with a stop contact in both directions of the axial movement so that the dose setting or display element can be moved in a defined manner up to a stop in one direction as far as a maximum dose position and in the other direction as far as a minimum dose position, e.g. the zero dose position.

In some embodiments, the injection device may have a force transmitting element, which biases the dose setting or display element with a force in one of the two axial directions. Although the injection device may be equipped with a force transmitting element, such a force transmitting element need not necessarily be provided, i.e. it is an optional force transmitting element. In some embodiments, the optional force transmitting element may generate the force needed to dispense a set or selected dose.

If no force transmitting element is provided, the user must apply the force needed to drive the plunger during the dispensing operation. In embodiments of this type, the dose setting or display unit may be extracted from the housing in the proximal direction when setting the dose and pushed backed deeper into the housing by the user during the dispensing operation. Optionally, an operating element moving out of the housing may act on the dose setting element by a gear during the dispensing operation.

In some preferred embodiments, when setting the dose, the dose setting or display element moves in the proximal direction and accordingly moves in the distal direction toward the injection site during the dispensing operation. This applies to embodiments which are not provided with a force transmitting element, namely if the dispensing operation is effected by applying axial pressure to the dose setting or display element or an operating element coupled with it by a gear mechanism. Although this operating sequence may be preferred for the dose setting or display element, the direction of movement for the setting operation and dispensing operation may just as easily be reversed if the force transmitting element is provided.

In accordance with some embodiments of the present invention, the dose setting or display element overlaps the reservoir, at least on assuming the at least one axial end position. If the injection device is provided with the optional force transmitting element, at least one of the dose setting or display element and the optional force transmitting element overlap the reservoir, at least when the dose setting or display element assumes the at least one axial end position. If the force transmitting element is provided, only the dose setting or display element overlaps the reservoir in such embodiments. In alternative embodiments, however, it is also possible for only the force transmitting element to be disposed in a position axially overlapping the reservoir. In yet another alternative, it is also possible for both the dose setting or display element and the force transmitting element be positioned axially overlapping the reservoir. If the force transmitting element overlaps, it may be axially supported on the housing, either directly or via an intermediate element, e.g. in the region of the overlap, in every axial position of the dose setting or display element. If, as in some preferred embodiments, the dose setting or display element overlaps, it does so in the axial end position assumed after dispensing the set dose.

In the at least one end position, the dose setting or display element or the optional force transmitting element overlaps or overlap not only a rear portion of the reservoir in relation to the forward drive direction, but advantageously also axially overlap the plunger. In some preferred embodiments, the dose setting or display element or the optional force transmitting element extend beyond the plunger in the forward drive direction, and in some preferred embodiments, at least do not locate behind the front end of the plunger.

Due to the overlap in the direction in which the dose setting or display element moves, the length of the injection device can be kept within ergonomically conducive limits, even in situations where the dose setting or display element is to be enabled to cover an axially long displacement path when setting the dose. From a tactile point of view, long displacement paths are beneficial for setting an exact dose if the dose is set by moving the dose setting or display element. Long displacement paths also offer an advantage on a purely display element because the dose scale can be distributed across a long distance corresponding to the displacement path.

If the dose setting or display element is a combined dose setting and display element in one, or if it constitutes a display element of the injection device and a pure or dedicated dose setting element is provided in addition, and the position it assumes after setting the dose defines the conveying stroke of the plunger, e.g. by a stop on reaching the minimum dose position, the dose setting and display element or the display element may serve as a support for the dose scale. The dose scale may alternatively be applied to or incorporated in the housing and the pure display element or the dose setting and display element serve as a support for a marking which migrates along the dose scale, for example in the form of a reading strip. In some preferred embodiments, the dose setting or display element combines the functions of setting the dose and providing a display, in which case the injection device of the present invention offers said advantages of the two functions in combination.

The dose setting or display element may move solely in translation, i.e. only axially. Advantageously, however, it can also be rotated about a rotation axis pointing in the axial direction. In some preferred embodiments, the translating and rotating movements are forcibly superimposed on one another, to which end the dose setting or display element forms a link element of a screw joint, the other link element of which may be formed by the housing. The two link elements sit in a threaded engagement with a thread pitch which is large enough so that an axial force applied to the dose setting or display element, e.g. a pressing force, when in the thread engagement causes a rotation of the dose setting and display element with a superimposed translating movement. The two engaged threads are therefore not self-locking. The dose setting or display element advantageously sits in a direct threaded engagement with the housing. Optionally, it is not coupled with the housing directly, but instead is coupled with it kinematically via one or more intermediate elements which can be displaced relative to the housing, i.e. slip-free. During the movement of the dose setting or display element, intermediate elements fixedly connected to the housing may be regarded as belonging to the housing as far as the movement of the dose setting or display element is concerned.

If the at least one end position is defined by a stop, as in some preferred embodiments, the stop maybe an axial stop against which the dose setting or display element presses in the axial direction on reaching the end position. If a rotating movement is superimposed on the axial movement, the stop may also be an axial stop or alternatively a radial or rotation stop, with which the dose setting or display element makes an abutting contact during the rotation in the circumferential direction about the rotation axis when it has reached the at least one end position.

In some embodiments, to provide a defined displacement of the dose setting or display element during the setting operation and also during the dispensing operation, the dose setting and display element is provided with a mating region in the form of a thread as described above, for example in the form of a linear guide with catch elements disposed axially adjacent to one another. In preferred embodiments wherein the dose setting or display element can be moved in an axially overlapping arrangement with the plunger, the mating region of the dose setting or display element also overlaps the plunger, advantageously up to at least its front end.

The force transmitting element, if provided, in some embodiments may be a spring element, which biases the dose setting element with an elastic force. This being the case, it may be a pneumatic or a mechanical spring element. In embodiments involving a spring element, the latter releases the elastic energy stored previously during the operation of setting the product dose during the dispensing operation and thus supplies the energy needed to drive the plunger forwards. The user only has to operate a trigger. Irrespective of the type of force transmitting element used, the end position in which the dose setting and display element overlaps the reservoir just or to the farthest point is the minimum dose position or the maximum dose position. If the force transmitting element is a compression spring, it acts on the dose setting or display element in the direction toward the minimum dose position (this direction coincides with the distal direction). If the compression spring is disposed distally of the dose setting or display element, its elastic force acts in the proximal direction. If the force transmitting element is a tension spring, for example, the directional relationship can be reversed. A situation where the actual force transmitting element has practically no relevant elasticity should also not be ruled out, and instead it may be formed by an electric motor, for example, which permits or also effects a dose setting movement of the dose setting element when the dose is being set or alternatively causes it by a motor driving action after a separately effected setting operation depending on the setting and also causes a dispensing movement of the dose setting element by a motor driving action when manually triggered.

In fulfilling the function of a dose setting element, the dose setting or display element may extend out of the housing so that it forms a manually operable dose setting knob itself, in which case the portion extending out of the housing may be formed integrally with the remaining part of the dose setting element or may be fixedly connected to it. In this case, the dose setting or display element moves out of the housing during the operation of setting the dose and is moved back into the housing in the direction toward the minimum dose position again during the dispensing operation, this advantageously being done by the user pushing on the dose setting or display element in the distal direction.

In some embodiments, if a force transmitting element is provided, the dose setting or display element does not move out of the housing and instead moves backward and forward inside the housing during setting and dispensing the dose.

In embodiments incorporating a force transmitting element, the dose setting or display element may be provided in the form of a dose setting coupling retained in the respective dose setting position relative to the housing against the force of the force transmitting element. The dose setting coupling comprises coupling elements, which are in a coupled engagement with one another when the dose is being set. The coupled engagement is released for the dispensing operation so that the dose setting or display element is able to move in the direction toward the end position under the force of the force transmitting element. If the dose setting or display element does not itself extend out of the housing or is fixedly connected to a dose setting knob which can gripped and held, the dose setting coupling provides a coupling between the dose setting or display element and a dose setting knob which is accessible to the user and can therefore be manually operated to set the dose. To dispense the set dose, the dose setting coupling is released and the dose setting or display element is uncoupled from the externally accessible dose setting knob as a result. To release the coupled engagement, the injection device has an operating knob serving as a trigger. In some preferred embodiments, the operating knob is disposed on a proximal end of the injection device and may advantageously be operated with the thumb of the same hand as that with which the user holds the injection device during the injection.

In some preferred embodiments the operating knob constitutes the proximal end of the injection device. It is also of advantage if the operating knob also serves as the dose setting knob and thus fulfils a dual function. In embodiments both without and with the force transmitting element, the same manually operable operating knob acts both as the dose setting knob and the trigger, operation of which causes the dispensing operation.

In some preferred embodiments, the injection device has a dispensing coupling, which provides a kinematic coupling between the dose setting or display element and a plunger for the dispensing operation, i.e. slip-free. During the operation of setting the dose, the coupled engagement of the coupling elements constituting the dispensing coupling is released so that the dose setting or display element is uncoupled from the plunger rod. In embodiments in which the injection device has a dose setting coupling for providing a coupling between the dose setting or display element and said dose setting knob during the operation of setting the dose and a dispensing coupling for providing a coupling between the dose setting or display element and the plunger rod during the dispensing operation, the two couplings are “switched” so that when triggered by operating said operating knob, the dose setting coupling is automatically released and the dispensing coupling closed.

### BRIEF DESCRIPTION OF THE DRAWINGS

FIG. 1 is a view of one embodiment of an injection device in accordance with the present invention;

FIG. 2 shows the injection device in longitudinal section;

FIG. 3 shows the proximal portion of the injection device in longitudinal section;

FIG. 4 shows an embodiment of a dose setting coupling in a coupled engagement;

FIG. 5 shows an embodiment of a housing portion and an operating knob; and

FIG. 6 shows an embodiment of a dispensing coupling out of engagement.

### DETAILED DESCRIPTION

With regard to fastening, mounting, attaching or connecting components of the present invention, unless specifically described as otherwise, conventional mechanical fasteners and methods may be used. Other appropriate fastening or attachment methods include adhesives, welding and soldering, the latter particularly with regard to the electrical system of the invention, if any. In embodiments with electrical features or components, suitable electrical components and circuitry, wires, wireless components, chips, boards, microprocessors, inputs, outputs, displays, control components, etc. may be used. Generally, unless otherwise indicated, the materials for making the invention and/or its components may be selected from appropriate materials such as metal, metallic alloys, ceramics, plastics, etc.

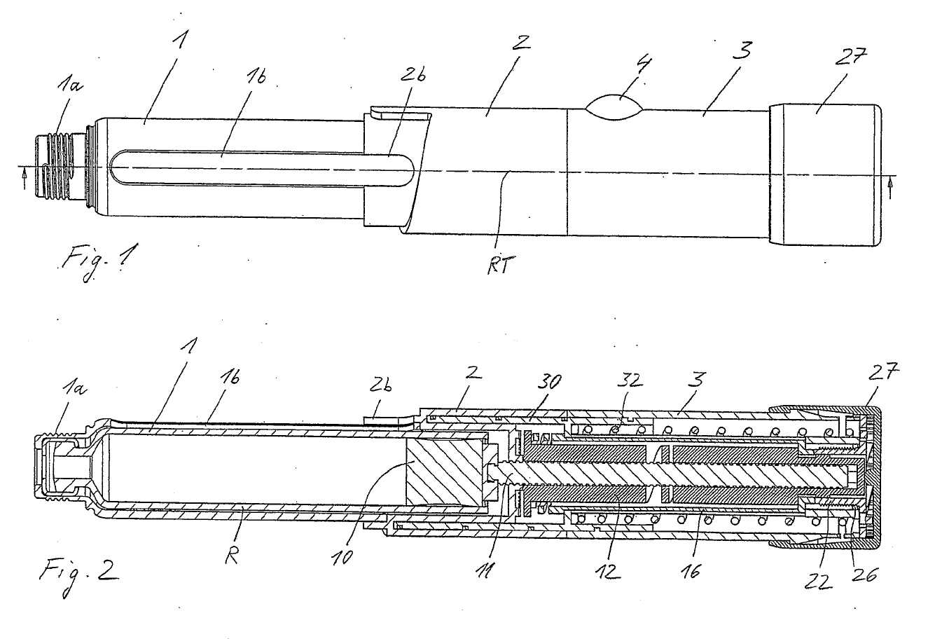

FIG. 1 shows a side view of an embodiment of an injection device in accordance with the present invention. The injection device is a longitudinally extending, slim injection pen. It comprises a housing with a distal (or front) housing portion 1 and a proximal (or rear) housing portion made up of two housing portions 2 and 3 fixedly connected to one another. The injection device is used to administer a liquid medicament, for example insulin. The medicament is contained in a reservoir, which can be replaced by inserting it in the housing portion 1. The medicament can be administered through an outlet 1a provided at the distal end of the housing portion 1. By reference to a central longitudinal axis RT, the injection device is essentially rotationally symmetrical. The housing portions 1-3 are each made from a non-transparent plastic material. To make it possible to see through to the reservoir, housing portion 1 has a window 1b, which extends up to or close to the proximal end of housing portion 1 and close to the outlet 1a, but is relatively slim in the circumferential direction about the axis RT. Housing portion 2 likewise has a window 2b, which overlaps the window 1b when housing portions 1 and 2 are connected, providing a free view through to the reservoir in the connecting region of housing portions 1 and 2. The windows 1b and 2b are of the same width in the circumferential direction, but could conceivably be of different dimensions in the circumferential direction, provided the function of providing a view through to the reservoir can be fulfilled. Another window 4 in the form of a magnifying glass is also provided in housing portion 3. An operating knob 27 forms the proximal end of the injection device. The operating knob 27 fulfils the function of a manually operable dose setting knob, which enables a dose to be selected or set for a medicament to be administered when operated and also serves as a trigger button enabling the set dose to be dispensed when operated. The set dose can be read through the window 4 on a dose scale extending continuously below the window 4 during the setting operation.

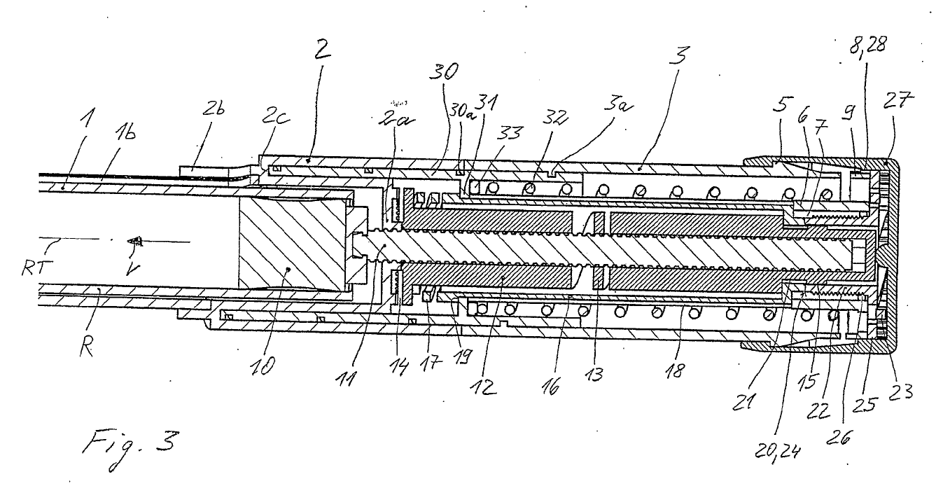

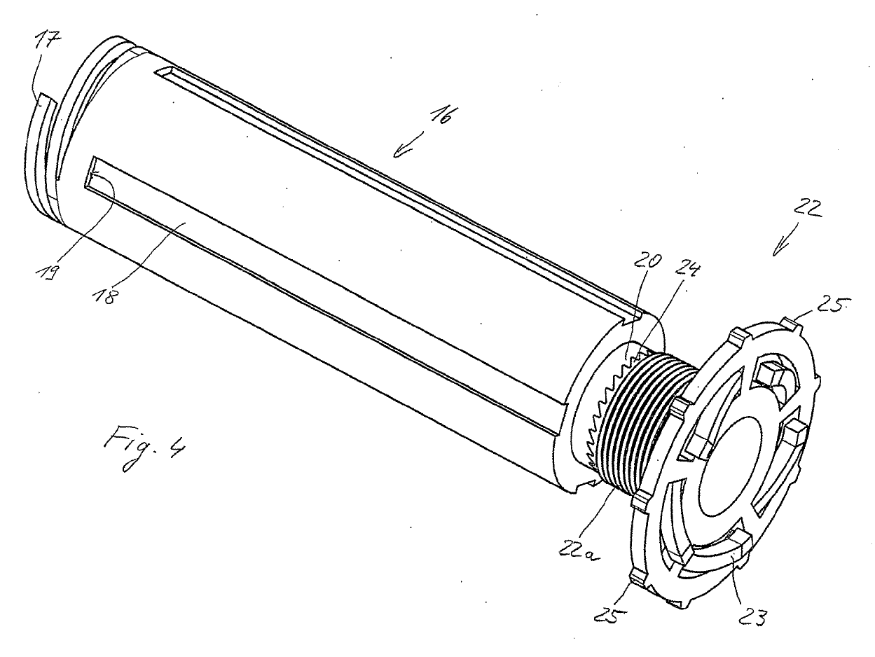

FIGS. 2 and 3 each illustrate the injection device in a longitudinal section containing the longitudinal axis RT, FIG. 2 showing it as a whole and FIG. 3 illustrating the proximal portion on a larger scale.

The reservoir R is an ampoule made from glass or transparent plastic. Disposed in the reservoir R is a plunger 10 which can be moved axially in a forward drive direction V toward the outlet 1a. In the initial state illustrated in FIGS. 2 and 3, the reservoir R is completely full and the plunger 10 closing off the reservoir R at the proximal end assumes its most proximal position. Basically, the housing portion 1 merely serves as a reservoir holder. It is fixedly connected to the housing portion 2 but can be released by screwing, for example, to enable the reservoir 1 to be replaced.

Portions 2 and 3 constituting the proximal housing portion are likewise fixedly connected to one another, e.g. by a material join, and may be regarded as a single housing portion in terms of their function. The housing portion formed by the two portions 2, 3 provides a mount for or carries a dose setting and forward drive mechanism, by which a dose of medicament can be set or selected for dispensing with each injection and the set dose can be dispensed by the forward driving action of the plunger 10. The operating knob 27 is coupled with the dose setting and forward drive mechanism for setting the dose by a dose setting coupling.

The dose setting and forward drive mechanism comprises several elements, which are coupled with one another in different ways by the dose setting coupling and a dispensing coupling when the dose is being set and dispensed. A plunger rod 11 is one of these elements. During the dispensing operation, the plunger rod 11 pushes against the rear face of the plunger 10 so that it moves in the forward drive direction V and dispenses medicament through the outlet 1a. The plunger rod 11 sits in a threaded engagement with the housing portion 2, for which purpose it is provided with a thread across the major part of its length. Housing portion 2 constitutes the co-operating thread on a retaining mechanism 2a (FIG. 3) projecting radially inward toward the plunger rod 11. The forward driving movement of the plunger rod 11 is a rotating movement about the longitudinal axis RT with a translating movement in the forward drive direction V superimposed on it. The thread of the plunger rod 11 is not continuous but is interrupted by an axially flat face or groove. However, this does not cause any interruption to the thread engagement with the retaining mechanism 2a and the plunger rod 11 retained in the thread engagement.

The dose setting and forward drive mechanism also has a first coupling element 12, a second coupling element 16 and a third coupling element 22. Coupling element 12 is able to move axially with the plunger rod 11 along its flat face or groove in a guiding engagement but is connected so that it is prevented from rotating. The coupling element 12 is disposed proximally of the retaining mechanism 2a and pushes against the retaining mechanism 2a in the forward drive direction V. The coupling element 12 surrounds the plunger rod 11. The coupling element 12 comprises a distal portion which sits in contact with the retaining mechanism 2a and a proximal portion which extends as far as the operating knob 27. A spring 13 is provided between the two portions. Another spring 14 is provided on the distal end of the coupling element 12, which has several resilient lugs which project in the direction toward the retaining mechanism 2a and are elastically biased. The resilient lugs of the spring 14 act as catch elements, which latch with catch elements of the retaining mechanism 2a, thereby providing an anti-rotation slip coupling between the coupling element 12 and the retaining mechanism 2a and hence housing portion 2, which prevents the plunger rod 11 from rotating relative to the housing 1-3 during dispensing operations. However, the force which can be transmitted when the catch elements and co-operating catch elements of the anti-rotation coupling is not so strong that it prevents the rotating movement of the plunger rod 11 needed to dispense the set dose or prevents it to only a practically relevant degree. In the portion of the spring 13, the coupling element 12 may flex axially. The coupling element 12 incorporating the two springs 13 and 14 is made integrally from plastic, for example by an injection moulding process.

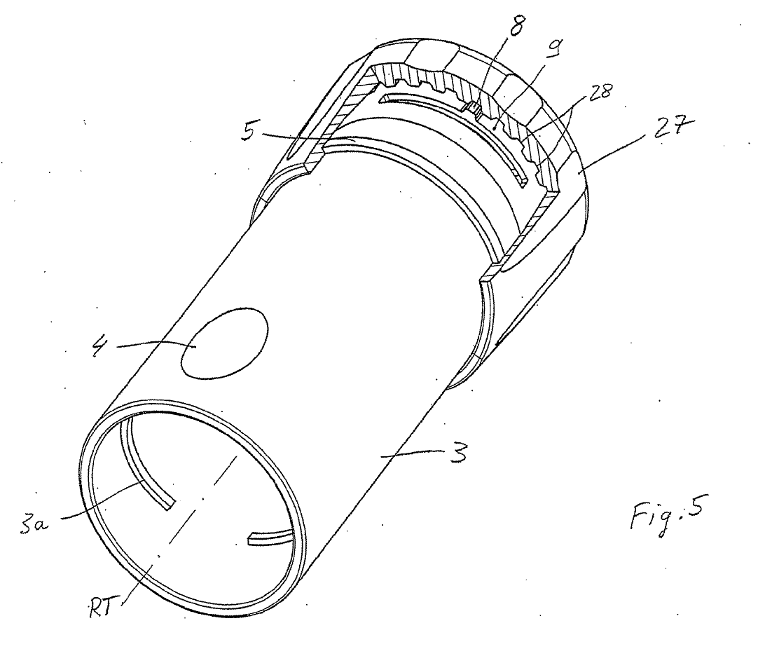

The coupling element 16 is mounted so that it can rotate about the axis RT. It is a generally cylindrical, sleeve-shaped body and surrounds the coupling element 12. Integrally formed on the distal end of the coupling element 16 is a spring 17, which in the embodiment illustrated as an example is an axially short helical spring. The coupling element 16 is supported by its spring 17 on the coupling element 12 in the distal direction and pushes it against the retaining mechanism 2a. Formed on the proximal end of the coupling element 16 are mating elements 20, which sit in a releasable coupled engagement with co-operating mating elements 24 of the coupling element 22 when the injection device is in the state illustrated. The mating elements 20 and co-operating mating elements 24 may be teeth projecting axially out from the coupling element 16 or 22 in the direction toward the other ones, which form two toothed rings locating with one another in the coupled engagement concentric with the axis RT, e.g. with a uniform tooth distribution, as illustrated by way of example in FIG. 4. In the state illustrated, in which the user is able to set the dose to be administered, the mating elements 20 and co-operating mating elements 24 of the two coupling elements 16 and 22 locate in one another. The coupling elements 16 and 22 constitute the dose setting coupling and are connected to one another so that they can not rotate in the coupled engagement.

FIG. 4 illustrates the coupling halves of the dose setting coupling, namely the coupling elements 16 and 22, in the coupled engagement and removed from the overall context so that their functional elements 17-20 on the one hand and 23-25 on the other hand are more readily visible.

The coupling element 22 is likewise sleeve-shaped and has a flange projecting radially outward at its proximal end. Formed on the external circumference of the flange are mating elements 25, which connect the coupling element 22 to the operating knob 27 so that it can not rotate. The coupling element 22 is axially supported on the operating knob 27 by a spring 23. The spring 23 is an integrated part of the coupling element 22 insofar as the coupling element 22 is made from plastic in one piece incorporating the spring 23. Like spring 14, spring 23 comprises several resilient lugs projecting axially out and bent about the axis RT. In conjunction with the spring 17, it ensures that the coupling elements 16 and 22 are elastically retained axially in the coupled engagement. The spring 17 pushes the coupling element 16 and the spring 23 pushes the coupling element 22 axially in each case into contact with the housing portion 3.

The coupling element 22 is in a threaded engagement with a stop element 26. To this end, the coupling element 22 is provided with a thread 22a (FIG. 4) on its external circumference before the flange. The housing portion 3 acts an axial guide 7 for the stop element 26 lying radially outward opposite the thread 22a so that the stop element 26 is moved axially in the threaded engagement when the coupling element 22 is moved in rotation. The axial displacement path of the stop element 26 corresponds to the quantity of medicament which is available as a maximum and can be administered in several injections, i.e. when the reservoir R is full. When the coupling element 22 is moved in rotation, the stop element 26 migrates, guided by the axial guide 7 along the external thread 22a of the coupling element 22, in the axial direction, which is the forward drive direction V in the embodiment illustrated as an example. Once the stop element 26 reaches an axial stop formed by the inner sleeve 6 in this direction of movement, it means that the reservoir R has been completely emptied. The inner sleeve 6 acts as the axial guide 7 and is not able to move axially relative to the housing portion 3, and, in the embodiment illustrated as an example, is formed on the housing portion 3 by a radial connecting web, i.e. is an integral part of the housing portion 3.

The dose setting and forward drive mechanism also has a dose setting and display element 30, which is in a threaded engagement with the housing portion 3. To provide this engagement, the housing portion 3 is provided with an internal thread 3a. Housing portion 2 is smooth in the contact region with the dose setting and display element 30. The thread 3a is disposed directly on the internal surface of the circular cylindrical sleeve shell of the housing portion 2, 3. The dose setting and display element 30 is essentially a simple circular cylindrical sleeve with an external thread 30a of complementary shape. The threads 3a and 30a have a significantly bigger, or larger or higher, thread pitch than the threads of the plunger rod 11 and retaining mechanism 2a. The thread pitch is large enough to prevent retention in the thread engagement by friction and the dose setting and display element 30 is rotated and moved axially under the effect of a purely axial force relative to the housing portion made up of two parts in the threaded engagement.

The dose setting and display element 30 may be moved axially but is coupled with the coupling element 16 so that it is prevented from rotating. In the embodiment illustrated as an example, the dose setting and display element 30 and the coupling element 16 sit directly in an appropriate guided engagement with one another. To this end, the coupling element 16 forms an axial guide 18 at its external circumference. A mating element 31 projects out from the internal face of the dose setting and display element 30, by which the dose setting and display element 30 is in a guided engagement with the coupling element 16, i.e. with its guide 18. The guide 18 extends across the major part of the length of the coupling element 16 and the dose setting and display element 30 travels through the major part of its length when setting the maximum dose. The guide 18 is formed by axial grooves in the external surface of the coupling element 16 (FIG. 4). At their distal ends, the grooves each serve as a stop 19 for the mating element 31.

The dose setting and forward drive mechanism comprises a spring element 32, which applies a force to the dose setting and display element 30 in the distal direction. The spring element 32, in the embodiment illustrated as an example, is a helical spring and acts as a compression spring. The spring element 32 is supported on the housing portion 3 in the proximal direction. In the distal direction, the spring element 32 is supported on an annular sliding disc 33, inserted between the coupling element 16 and the dose setting and display element 30 and is in turn supported on the mating element 31. By the sliding disc 33, the spring element 32 is largely uncoupled from any rotating movements of the dose setting and display element 30.

The dose setting and display element 30 is able to move backward and forward relative to the plunger 10 and the plunger rod 11 and relative to the coupling elements 12, 16 and 22 between axial end positions. The two end positions are a zero dose position and a maximum dose position. The two end positions are each predefined by a stop. The stop for the zero dose position is formed by a shoulder 2c at the distal end of the housing portion 2 and serves as an axial stop. The stop for the maximum position is formed by the housing portion 3. In the zero dose position, a distal portion of the dose setting and display element 30 overlaps the reservoir R in the forward drive direction V to beyond the plunger 10 when the plunger 10 assumes a rearmost position in the reservoir R as illustrated in FIGS. 2 and 3. The plunger 10 assumes this position when the reservoir R is completely full. The thread 30a extends as far as the distal end or at least close to the distal end of the dose setting and display element 30 so that the thread 30a also axially overlaps the reservoir R and the plunger 10 when the dose setting and display element 30 assumes the zero dose position. The thread 30a terminates before the distal end of the dose setting element 30 and in conjunction with the thread 3a of the housing portion 3 forms an anti-rotation stop, which defines the maximum dose position of the dose setting and display element. Alternatively, the thread 30a could also terminate at the distal end of the dose setting and display element 30; the stop defining the maximum dose position would then be of a different design in the event of such a modification.

At its distal end, the dose setting and display element 30 is not circumferentially completely cylindrical but part-cylindrical so that, in spite of the axial overlap, the view onto the plunger is exposed if the dose setting and display element 30 is not made from a transparent plastic material, although this is preferred, but is made from a non-transparent or opaque plastic material or some other material. The distal edge of the dose setting and display element 30 extends in a spiral shape about the axis RT so that the dose setting and display element 30 stands out axially by its distal end in a circumferential region, even beyond the plunger 10, as may be seen from FIGS. 2 and 3, and sits back behind the plunger 10 in another circumferential region overlapping the window in the zero dose position so that the plunger 10 can be observed through the windows 1b and 2b. Instead of a spiral-shaped edge, the dose setting and display element 30 may extend circumferentially up to the same axial height all round with the exception of an interrupted region, for example, and thus extends across the plunger 10 in the forward drive direction V with the exception of the interrupted region, in which case the interruption would overlap with the window 2b in the zero dose position. The shoulder 2c of the housing portion 2 likewise winds about the axis RT following the spiral path of the distal edge of the dose setting and display element 30, likewise winding about the axis RT. The winding course of the shoulder 2c may also be seen in FIG. 1. The housing portion 2 has an outer sleeve, which encloses the dose setting and forward drive mechanism in conjunction with the housing portion 3, and an inner sleeve which is connected to the outer sleeve at its distal end by a web forming the shoulder 2c, and the proximal end of which projects radially inward toward the retaining mechanism 2a. An annular gap is left free between the outer and the inner sleeve, through which the dose setting and display element 30 extends across the greater part of its length in the zero dose position, and the greater part in the region of the longest circumferential segment of the dose setting and display element 30 amounts to approximately 50% of the total length of the dose setting and display element 30.

The dose setting and display element 30 provides a support for a dose scale, which extends round in a spiral on the external circumference of the dose setting and display element 30 with a pitch which corresponds to the pitch of the thread 30a as measured by reference to the axis RT. The dose scale is made up of markings and numbers, each of the markings corresponding to a smallest dose unit which can be set. During the operation of setting the dose to be administered, the dose scale can be read through the window 4 of the housing portion 3 (FIG. 1).

The operating knob 27 forms the proximal end of the injection device. It clips onto the housing portion 3 by a shoulder 5 provided on the proximal end of the housing portion 3, which grips behind the operating knob 27. The operating knob 27 is able to rotate relative to the housing 1-3 about the axis RT in a dose setting direction and a correction direction. When setting the dose, the dose is increased by rotating the operating knob 27 in the dose setting direction and reduced in a rotating movement in the correction direction, thereby enabling a dose that has been accidentally set too high to be corrected. The operating knob 27 is also axially displaceable with the coupling element 22 but is connected so that it is prevented from rotating by the mating elements 25.

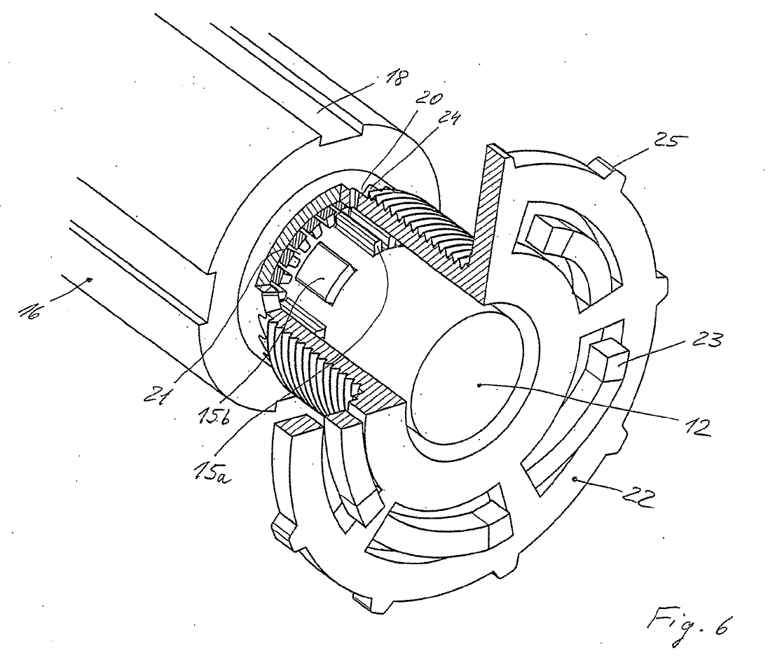

FIG. 5 illustrates the housing portion 3 and the operating knob 27 in the coupled engagement, with part of the operating knob cut away to illustrate the engagement.

Due to the anti-rotation lock with the coupling element 22, the operating knob 27 forms a slip coupling, in the embodiment illustrated as an example, an anti-rotation slip coupling, with the housing portion 3. Several catch elements 28 are disposed on the operating knob 27, uniformly distributed about the axis RT forming the slip coupling, which sit in a coupled engagement with co-operating complementary catch elements 8 preferably congruently disposed on the distal end of the housing portion 3. The number of complementary catch elements 8 is lower than the number of catch elements 28. When the coupled engagement is established between the catch elements 28 and complementary catch elements 8, the operating knob 27 can be rotated relative to the housing portion 3 about the axis RT, and the catch elements 28 and complementary catch elements 8 are able to latch with one another in a releasable arrangement in pairs distributed discretely about the circumference. The pitch of the catch elements 28 and hence the distance as measured in the angle or arc dimension between the respective adjacently disposed catch elements 28 in the circumferential direction corresponds to the smallest dose unit which can be set and the pitch of the complementary catch elements corresponds to a whole number multiple of the same dose unit. The slip coupling need not necessarily be disposed directly between the housing 1-3 and the operating knob 27. Instead of providing the catch elements 28 on the operating knob 27, they could also be disposed on the coupling element 22. Modified catch elements and complementary catch elements could also locate in one another axially rather than radially in both embodiments.

The catch elements 28 and accordingly the complementary catch elements 8 are of an asymmetrical shape with reference to the direction of rotation so that the force needed to release the catch engagement in a coupling dose setting direction is stronger than in the opposite direction of rotation, namely the coupling correction direction. Since the catch elements 28 are disposed directly on the operating knob 27 in the exemplary embodiment, the dose setting direction of the operating knob 27 is simultaneously the coupling dose setting direction, and the correction direction is the coupling correction direction. The complementary catch elements 8 are catch cams projecting radially outward from the external surface of housing portion 3. Accordingly, the catch elements 28 are provided in the form of co-operating recesses or tooth gaps of internal teeth on the facing, opposite internal surface of the operating knob 27. To obtain the asymmetry for the two directions of rotation, the leading edges of the catch elements 28 pointing in the coupling dose setting direction as the catch elements move 28 are flatter than the trailing edges pointing in the opposite direction of rotation. The two edges of the complementary catch elements 8 are likewise shaped accordingly.

The asymmetry of the slip coupling by reference to the two rotation directions of the operating knob 27 is adapted to the direction of the force expended by the force transmitting element 32 on the dose setting and display element 30. As the operating knob 27 is moved in the direction for increasing the dose, the dose setting and display element 30 is rotated in the thread engagement due to the dose setting coupling formed at 20, 24 and the guide coupling formed at 18 and 31, and moves in the proximal direction. During the course of the translating movement in the proximal direction, the spring element 32 is tensed to an increasing degree. The elastic force of the spring element 32 produces a resistance countering the movement of the dose setting and display element 30 in the proximal direction, which acts on the slip coupling due to the coupling described above, and an additional frictional resistance which increases as the dose is increased counteracts the rotating movement of the operating knob 27 in the dose setting direction, in which the flatter edges of the catch elements 28 and complementary catch elements 8 point. Conversely, the force expended by the spring element 32 assists the rotating movement in the direction of a dose correction.

FIG. 5 illustrates the housing portion 3 and its complementary catch elements 8 for the slip coupling. The housing portion 3 forms coupling springs 9 which are radially elastically flexible in a proximal sleeve portion, namely one coupling spring 9 per complementary catch element 8. The complementary catch elements 8 respectively project radially outward from the coupling springs 9. The coupling springs 9 are circle segments. The sleeve portion comprises the coupling springs 9 extending about the axis RT, alternating with circle segments which are stiffer than them. The coupling springs 9 respectively act as bending beams extending in the circumferential direction which are biased on both sides, namely on the two respective stiffer circle segments disposed closest. Extending in the circumferential direction along the coupling springs 9 is a respective recess, thereby increasing the flexibility of the respective coupling spring 9. The sleeve portion incorporating the coupling springs 9 forms the proximal end of the housing portion 3.

The operating knob 27 is pot-shaped with a base constituting the distal end of the injection device and a wall projecting out from the base extending about the axis RT, on the internal face of which the catch elements 8 are uniformly distributed about the circumference and each shaped as an axially extending recess. In the assembled state, the catch elements 28 engage with both the complementary catch elements 8 and the mating elements 25 (FIG. 3) of the coupling element 22, as a result of which both the catch engagement with the complementary catch elements 8 and the anti-rotation engagement with the mating elements 25 are maintained in every axial position of the operating knob 27.

The dose setting and forward drive mechanism contains a second coupling due to the dose setting coupling formed by the coupling elements 16 and 22, namely the dispensing coupling already mentioned above, which is illustrated in FIG. 6. The dispensing coupling comprises the coupling elements 12 and 16, which are provided with mating elements which engage with one another in the coupled engagement for this purpose. These are mating elements 15a and 15b on the side of the coupling element 12. The coupling element 16 is provided with mating elements 21. The mating elements 15a are axial ribs which project radially outward from an internal surface of the coupling element 12 at the proximal end. The mating elements 21 are provided in the form of co-operating axially blind grooves on an internal surface at the proximal end of the coupling element 16. The mating elements 21 form an axial guide for the mating elements 15a. The mating elements 15a constitute a sort of external teeth or toothing about the axis RT. The external teeth are interrupted at one point at least and, in the embodiment illustrated as an example, at two points lying diametrically opposite one another. Disposed in each interrupted circumferential region is one of the mating elements 15b. Like the mating elements 15a, the mating elements 15b project radially outward but are wider than the mating elements 15a in the circumferential direction so that they are not able to move into the mating elements 21 formed by blind grooves. Instead, the rib webs left between the mating elements 21 form an axial stop for the mating elements 15b when the coupling element 12 is moved in the distal direction relative to the coupling element 16.

The coupling element 16 is moved selectively with the coupling element 22 or coupling element 12 in the coupled engagement, i.e. in the coupled engagement with the coupling element 22 when the dispensing coupling is released and in the coupled engagement with the coupling element 12 when the dose setting coupling is released. To set the dose, the dose setting coupling is closed, i.e. the mating elements 15a and 15b axially overlap only the coupling element 22, the internal surface of which is circumferentially smooth so that the coupling element 22 is able to rotate relative to the coupling element 12 as the dose is being set. To dispense the set dose, the coupling element 12 moves into the coupled engagement with the dose setting and display element 16, the dose setting coupling is released as a result and the dispensing coupling is closed so that the dispensing operation can start. FIG. 6 illustrates the coupling elements 12, 16 and 22 in the state in which the dose setting coupling is closed.

The user holds the injection device in one hand and places a cannula unit on the outlet 1a and screws it tight with the other hand. Otherwise, the injection device is in the state illustrated in FIGS. 1 to 3. Due to the windows 1b and 2b, the user is able to see how full the reservoir R is and check the position of the plunger 10. It is assumed the reservoir has been primed and that the user would like to inject a specific dose of medicament in the next step. The desired dose is set by turning the operating knob 27. During the setting operation, the user can take a reading of the dose corresponding to the axial position of the dose setting and display element 30 at any time through the window 4. If a dose is accidentally set too high during the process of increasing the dose, the user can correct the overdose by turning the operating knob 27 in the correction direction. During the process of setting the dose, the rotating movement of the operating knob 27 is transmitted to the coupling element 22 via the extant anti-rotation lock at 25 and to the coupling element 16 via the closed dose setting coupling at 20, 24 and to the dose setting and display element 30 in the guide engagement between 18 and 31. With the threads 3a and 30a in the engaged state, the dose setting and display element 30 moves in rotation about the axis RT and in translation in the proximal direction. As the dose is being increased, the spring element 32 becomes increasingly elastically tensed and assists any dose correction which might be needed. Both when increasing the dose and also correcting the dose, the catch engagement of the slip coupling between the housing portion 3 and the operating knob 27 makes a clearly perceptible clicking noise. Due to the steepness of the threads 3a and 30a, the dose setting and display element 30 travels a correspondingly long displacement path in the axial direction. The distances between the dose marks of the dose scale of the dose setting and display element 30 are equally long in the axial direction, thereby providing a clear reading of the dose marks extending below the window 4, even for users with a impaired sight.

The plunger rod 11 is uncoupled from the dose setting movement which, in the embodiment illustrated as an example, is the dose rotating movement, and is additionally prevented from effecting any rotating movements due to the surrounding coupling element 12, which is likewise uncoupled. As explained above, the lock established by the slip coupling produced between the coupling element 12 and retaining mechanism 2a and the anti-rotation engagement of the coupling element 12 with the plunger rod 11 are maintained.

When the desired dose has been set, the user pierces the desired injection site with the piercing cannula and moves it into the subcutaneous tissue below the skin. With the same hand which he is using to hold the injection device during piercing, he then triggers dispensing of the set dose.

The operating knob 27, which fulfils the function of a dose setting knob whilst the dose is being set, serves a dual function and is also the trigger knob. When the dose is being set, the dose setting and display element 30 is retained depending on the dose catch positions of the catch elements 28 and complementary catch elements 8 of the slip coupling, i.e. the slip coupling prevents the dose setting and display element 30 from being able to move under the action of the spring element 32. For the dispensing operation, therefore, the coupling between the housing portion 3 and the dose setting and display element 30 established via the slip coupling at 8, 28 must be released. This is done by releasing the dose setting coupling established at 20, 24, for which purpose the user presses the operating knob 27 in the distal direction with the thumb. When an appropriate pressing force is applied, the operating knob 27 moves relative to the housing portion 3 and coupling element 22 against the force of the spring 23 in the distal direction and during this movement pushes against the coupling element 12. The coupling element 12 flexes axially inwards in the region of its integrated spring 13 so that its mating elements 15a move into engagement with the mating elements 21 of the coupling element 16 preventing any rotation. In a transition phase of the axial movement, the dose setting coupling between the coupling element 22 and the coupling element 16 is still closed, whilst the coupled engagement of the dispensing coupling between the coupling element 12 and coupling element 16 is also already being established. As soon as the mating elements 15b make contact with the axial stop of the coupling element 12 in the distal direction, however, continuing pressure on the operating knob 27 causes the coupling element 16 to move against the force of its spring 17 in the distal direction, and thus lift out of the coupled engagement with the coupling element 22.

As soon as the dose setting coupling has been released, the dose setting and display element 30 is screwed in the distal direction due to the elastic force of the spring element 32. The rotating element of the movement effected by the dose setting and display element 30 is transmitted to the coupling element 16 in the coupled engagement between the guide 18 and mating element 31. Since the dispensing coupling is closed, the coupling element 16 transmits the rotating movement to the coupling element 12, which in turn is connected to the plunger rod 11 so as to rotate in unison with it, so that the plunger rod 11 rotates in the thread engagement with the retaining mechanism 2a and pushes the plunger 10 in the forward drive direction V. The dispensing movement of the components involved in this operation is terminated when the dose setting and display element 30 makes contact with the shoulder 2c. The stroke or axial element of the displacement path of the dose setting and display element 30 therefore determines the stroke of the plunger 10 and hence the dispensed dose. Due to the fact that the thread pitch of the threads 3a and 30a is bigger than the threads of the plunger rod 11 and retaining mechanism 2a, the stroke of the dose setting and display element 30 is reduced to the stroke of the plunger 10 in accordance with the reduction ratio. During the dispensing movement, the slip coupling between the spring 14 and retaining mechanism 2a generates a clearly perceptible clicking noise, providing an acoustic indication to the user that medicament is being dispensed. The dispensing operation can also be at least generally checked through the windows 1b and 2b on the basis of the axial position of the plunger 10.

When the dose is being set, the stop element 26 in the thread engagement with coupling element 22 likewise moves as a function of the set dose. When the quantity of medicament predefined by the maximum stroke of the stop element 26 has been totally administered, which will not be the case until after several injections if the reservoir R was completely full, the user replaces the empty reservoir R with a new, full reservoir R. To this end, she merely has to separate housing portion 1 from the proximal housing portion (made up of portions 2, 3), insert the new reservoir R with the plunger 10 already accommodated in it and connect the front and rear housing portions to one another again.

In the exemplary embodiment, all the springs with the exception of the force transmitting element 32 are an integral part of a respective coupling element comprising a coupling part and spring part. Alternatively, however, one or more springs may also be provided separately from the respective coupling element in a conventional manner in the form of steel springs. This being the case, springs 13, 17 and 23 may just as easily be replaced by steel springs because they fulfil exclusively a spring function. To replace the spring 14, a replacement spring and catch elements on the distal end of the coupling element 12 could be provided.

As far as the disposition of the dose setting and display element 30 and spring element 32 is concerned, it should be pointed out that, instead of the dose setting and display element 30, the spring element 32 may be disposed axially overlapping the reservoir R as an alternative, in which case it could be supported on a thrust bearing, for example the shoulder 2c. If the fittings were reversed in this manner, the dose setting and display element 30 would be moved in the proximal direction until it made contact with a stop during the dispensing operation. In yet another alternative, the spring element 32 could be provided in the form of a tension spring, although a fitting incorporating a compression spring is more particularly preferred. Yet another alternative is described in German patent applications No. 10 2005 043 806.7 and 10 2005 043 807.5. As described in these applications, the spring element adapted to the slip coupling is provided in the form of a torsion spring. The older applications are incorporated herein by reference in connection with the adapted arrangement comprising the slip coupling and spring element.

Embodiments of the present invention, including preferred embodiments, have been presented for the purpose of illustration and description. They are not intended to be exhaustive or to limit the invention to the precise forms and steps disclosed. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An injection device comprising:
a) a housing with a reservoir for an injectable product,
b) a plunger axially displaceable in the reservoir in a forward drive direction,
c) a dose setting and forward drive mechanism for the plunger disposed in an axial extension of the reservoir, and
d) a dose setting and display element which is movable in an axial direction relative to the plunger to an end position, said dose setting or display element axially overlapping the reservoir at least when the dose setting or display element is disposed in the end position.

2. The injection device as claimed in claim 1, wherein the dose setting and display element at least partially axially overlaps the plunger when the dose setting and display element assumes the end position and the plunger assumes a rear axial position in the reservoir with reference to the forward drive direction.

3. The injection device as claimed in claim 2, wherein the dose setting and display element partially surrounds the plunger.

4. The injection device as claimed in claim 3, wherein the dose setting and display element has a front edge winding in a screw shape about a central longitudinal axis with reference to the forward drive direction, by which it partially overlaps the plunger in the circumferential direction when the dose setting and display element assumes the end position and the plunger assumes a rear axial position in the reservoir.

5. The injection device as claimed in claim 4, wherein the housing affords a view through to the plunger at least in a circumferential region which radially overlaps a viewing region on a front end of the dose setting and display element when disposed in the end position.

6. The injection device as claimed in claim 5, wherein the dose setting and display element comprises readable dose marks which can be read through the housing.

7. The injection device as claimed in claim 1, wherein the housing has a shoulder axially overlapping the reservoir which acts as a stop for the dose setting and display element defining the end position.

8. The injection device as claimed in claim 7, wherein the housing forms two coaxial annular walls axially overlapping the reservoir and a gap between the annular walls with the shoulder as a base.

9. The injection device as claimed in claim 1, wherein the dose setting and display element is in a threaded engagement with a screw joint element and can be moved axially backward and forward in the threaded engagement.

10. The injection device as claimed in claim 9, wherein the threads establishing the threaded engagement have a thread pitch which causes a relative rotation between the dose setting and display element and the screw link element when subjected to axial pressure.

11. The injection device as claimed in claim 1, further comprising a force transmitting element and a mechanical spring subjected to pressure at least axially.

12. The injection device as claimed in claim 1, further comprising a force transmitting element which causes the forward driving movement of the plunger during dispensing via the dose setting and display element.

13. The injection device as claimed in claim 1, wherein the dose setting and forward drive mechanism comprises a plunger rod for driving the plunger forward and a dispensing coupling which uncouples the plunger rod from the dose setting and display element for setting the dose and couples it with the dose setting and display element for the dispensing operation.

14. The injection device as claimed in claim 13, wherein the dose setting and display element can be moved backward and forward relative to the reservoir along an axis in translation and about the axis in rotation, and the dispensing coupling couples the dose setting and display element to rotate in unison with the plunger rod for the dispensing operation.

15. The injection device as claimed in claim 1, further comprising a force transmitting element, wherein the dose setting and forward drive mechanism comprises a plunger rod for driving the plunger forwards and a dispensing coupling which uncouples the plunger rod from the force transmitting element for setting the dose and couples it with the force transmitting element for the dispensing operation.

16. The injection device as claimed in claim 13, wherein the dispensing coupling comprises a coupling element which can be moved axially with the dose setting and display element and is prevented from rotating, and which can be moved into a coupled engagement for dispensing the dose in which it couples the dose setting and display element with the plunger rod.

17. The injection device as claimed in claim 16, wherein the coupling element comprises a coupling part to provide the coupling with the dose setting and display element preventing rotation and a spring which is part of the coupling part and elastically biases the coupling element in a releasable coupled engagement in which the coupling element is non-rotatingly coupled with an operating knob which can be manually operated for setting a dose.

18. The injection device as claimed in claim 13, wherein the dispensing coupling comprises a coupling element coupled with the plunger rod so that it can move axially and is prevented from rotating, which can be moved into a coupled engagement for dispensing the dose in which it couples the dose setting and display element with the plunger rod.

19. The injection device as claimed in claim 1, further comprising a coupling element coupled with the plunger rod so that it moves axially but is rotationally locked and elastically coupled with the housing to be prevented from rotating.

20. The injection device as claimed in claim 19, wherein the coupling element comprises a coupling part for the rotationally locked coupling with the plunger rod and a spring which elastically biases the coupling element into a releasable anti-rotation engagement with the housing and is made in a single piece with the coupling part.

21. The injection device as claimed in claim 1, further comprising an operating knob which can be moved backward and forward relative to the housing in a dose setting direction and a direction opposite to the dose setting direction for setting the dose in a rotating movement, and a slip coupling with catch elements and complementary catch elements which when latched with one another couple the operating knob in discrete catch positions with the housing in a positive and non-positive arrangement and are of an asymmetrical shape with reference to the dose setting direction and the direction opposite the dose setting direction so that they afford a greater resistance to the movement in the direction opposite the dose setting direction than in the dose setting direction in the latched engagement.

22. The injection device as claimed in claim 21, further comprising a force transmitting element counteracting the movement of the operating knob in the dose setting direction.

23. The injection device as claimed in claim 1, further comprising a manually operable operating knob which can be moved, by a rotating movement, backward and forward relative to the housing for setting the dose, and is kinematically coupled with the dose setting and display element to be prevented from rotating.

24. The injection device as claimed in claim 23, wherein the operating knob is disposed on a proximal end of the injection device and triggers the dispensing operation when moved in an operating direction.

25. The injection device as claimed in claim 24, wherein the operating knob acts on a coupling element of the dispensing coupling when moved in the operating direction and moves the coupling element into a coupled engagement in which the dose setting and display element is coupled with the plunger rod.

26. The injection device as claims in claim 25, wherein the dose setting and forward drive mechanism comprises a plunger rod for driving the plunger forward and a dispensing coupling which uncouples the plunger rod from the dose setting and display element for setting the dose and couples it with the dose setting and display element for the dispensing operation.

27. The injection device as claimed in claim 25, further comprising a force transmitting element and wherein the dose setting and forward drive mechanism comprises a plunger rod for driving the plunger forward and a dispensing coupling which uncouples the plunger rod from the force transmitting element for setting the dose and couples it with the force transmitting element for the dispensing operation.

28. The injection device as claimed in claim 1, further comprising a manually operable operating knob which can be moved in rotation relative to the housing for setting the dose, and a dose setting coupling which couples the operating knob in a releasable coupled engagement with the dose setting and display element so that it can not rotate.

29. The injection device as claimed in claim 28, wherein the operating knob releases the coupled engagement of the dose setting coupling when moved in the operating direction due to the movement of the same coupling element on which the operating knob acts to produce the coupled engagement of the dispensing coupling.

30. An injection device comprising:
a housing with a reservoir for an injectable product;
a plunger axially movable in the reservoir in a direction of propulsion:
a dosing and propulsion driver provided for the plunger and arranged in an axial continuation of the reservoir;
an element, comprising at least one of a dosing member and display member, said element moveable in one axial direction relative to the plunger when setting a product dose and in the opposite direction when the dose is dispensed; and
a force-imparting member which forces the element in the direction of an axial end position, wherein at least one of the element and force-imparting member axially overlaps the reservoir, at least when the element is in the end position.

31. The injection device according to claim 30, further comprising a manually operable operating knob which can be moved, by a rotating movement, backward and forward relative to the housing for setting the dose, the knob being kinematically coupled to the element to be prevented from rotating.
